(19) United States
(12) Patent Application Publication
(10) Pub. No.: US 2005/0107310 A1
(43) Pub. Date: May 19, 2005

(54) Carboxylic acid glycuronides, glycosamides and glycosides of quinolones, penicillins, analogs, and uses thereof

(76) Inventors: Michael F Holick, Sudbury, MA (US); Halasya Ramanathan, Mysore (IN)

Correspondence Address: STERNE, KESSLER, GOLDSTEIN & FOX PLLC, 1100 NEW YORK AVENUE, N.W., WASHINGTON, DC, 20005, US

(21) Appl. No.: 10/508,140

(22) PCT Filed: Mar. 19, 2003
(86) PCT No.: PCT/US03/08260

Related U.S. Application Data
(60) Provisional application No. 60/365,180, filed on Mar. 19, 2002.

(51) Int. Cl. A61K 31/7052; C07H 17/02

(52) U.S. Cl. 514/23; 536/17.4

(57) ABSTRACT

Disclosed are caboxylic acid glycuronides, glycosamides and glycosides of quinolones, penicillins and analogs thereof to treat conditions and diseases such as bacterial infections.

### BACKGROUND OF THE INVENTION

1. Field of the Invention

The present invention relates to carboxylic acid glycosamides and glycosides of quinolones and penicillins and analogs thereof.

2. Related Art

Ofloxacin is a broad spectrum antibiotic of the quinolone class. See U.S. Pat. No. 4,382,892, which discloses ofloxacin and analogs thereof. Other drugs which are in the class of quinolones include ciprofloxacin, enoxacin, gatifloxacin, levofloxacin, lomefloxacin, moxifloxacin, norfloxacin, ofloxacin, pefloxacin, sparfloxacin, nalidixic acid and trovafloxacin. The penicillin group of antibiotics also contain a carboxyl group. In particular, amphicillin, carbenicillin and penicillin-G belong to the beta lactan antibiotics containing a carboxylic acid group.

### SUMMARY OF THE INVENTION

The present invention relates to a pro-drug approach to quinolone and beta lactam group of drugs that provides better bioavailability. The pro-drug is in the form of carboxylic acid glycuronides, glycosamides and glycosides of compounds belonging to the drug class often referred to as quinolones. Similar pro-drug approach can be extended to beta lactam group of antibiotics utilizing the carboxylic acid group. The carboxy group may be amidated with a protected amino sugar to give a glycosamide. Alternatively, the carboxy group may be esterified with a protected glycuronic acid to give a glycuronic acid ester or with a protected glycose to give a carboxylic acid glycose ester. The protecting groups are then partially or completely removed. When administered, the pro-drug targets the pathogen, especially bacteria and fungi. The cell surface of the bacterial walls exhibit enhanced glycosamine expression than the humans. Reference: Bacterial pathogenesis, A molecular approach by Abigai A. Salyers and Dixie D. Whitt, ASM press, Washington D.C. (1994). Thus, the compounds of the invention may have the advantage of targeting the pathogen more effectively.

The amidase and glycosidase enzymes present within the pathogen will liberate the drug causing the cell specific damage that is intended. Also when administered, glycosidase, amidase and esterase enzymes in the biological medium of human body cleave the ester/amide bonds, thus liberating the free drug. Thus, the free drug is bioavailable in a controlled fashion as determined by the rate of de-amidation/de-esterification. The compounds of the invention can be used for the treatment of any condition treatable by quinolones and penicillins including bacterial infections.

The present invention relates in particular to compounds of the Formula (I):
A-R′ (I)
wherein A is the residue of a quinolone, penicillin or analog thereof and R′ represents the residue of glycuronic acid, glycosamine or glycoside.

The present invention also relates to compounds having Formula (II): 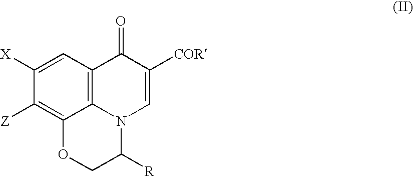
wherein X represents a halogen atom, R represents a hydrogen atom or an alkyl group having from 1 to 6 carbon atoms, R′ represents the residue of glycuronic acid, glycosamine or glycoside, and Z represents a mono-substituted amino group, a di-substituted amino group, or a cyclic-substituted amino group which may contain another hetero-atom, and the substituted amino group may be further substituted with one or more substituents selected from the group consisting of hydroxyl, alkyl having from 1 to 6 carbon atoms, amino, hydroxyalkyl having from 1 to 6 carbon atoms, monoalkylamino and dialkylamino having from 1 to 6 carbon atoms in each alkyl moiety, and pharmaceutically acceptable salts thereof. Preferably, R′ is a straight or branched chain glycosidic residue containing 1-20 glycosidic units per residue, or a glucuronic acid or glucosamine residue.

The invention also relates to a method for the treatment or amelioration of any condition treatable with quinolones, penicillins and analogs thereof, comprising administering to an animal in need thereof, an effective amount of a compound of the invention, or a pharmaceutically acceptable salt thereof.

The invention also relates to a method of preparing a compound of Formula (I) which comprises condensing a protected saccharide, aminosaccharide or glycuronic acid with a quinolone, penicillin or analog thereof, wherein said quinolone, penicillin or analog thereof that has a carboxy group, in solvent, and isolating the protected carboxylic acid glycuronide, glycosamide or glycoside ester. The protecting groups may then be partially or completely removed.

### DETAILED DESCRIPTION OF THE INVENTION

Where the derivative is a glycose ester, then it is preferred that it contain 1-20 glycosidic units.

It is preferred that compounds of the present invention have less than 10 and, more preferably, 3 or less glycosidic units. Specific examples are those containing 1 or 2 glycosidic units in the glycoside residue, such as glucose and sucrose, with one being most preferred.

By glycosidic units are meant glycopyranosyl or glycofuranosyl, as well as their sulfates and/or deoxy derivatives. The configuration of each unit may be D or L, although D is generally preferred. The residues may be homopolymers, random or alternating polymers, or block copolymers of these monomers.

The glycosidic units have free hydroxy groups, or the hydroxy groups may be acylated, e.g. with a group R₄—(C═O)—, wherein R₄ is hydrogen, C₁₋₆ alkyl, C₆₋₁₀ substituted or unsubstituted aryl or C₇₋₁₆ aralkyl. Preferably, the acyl groups are acetyl or propionyl. Other preferred R₄ groups are phenyl, nitrophenyl, halophenyl, lower alkyl substituted phenyl, lower alkoxy substituted phenyl and the like or benzyl, lower alkoxy substituted benzyl and the like.

The glycopyranose or glycofuranose ring or amino derivative thereof may be fully or partially acylated or completely deacylated. The completely or partially acylated glycoside is useful as a defined intermediate for the synthesis of the deacylated material. Useful protecting groups include, but are not limited to, acetyl, benzoyl, nicotinoyl, benzyl, methyl and phenyl.

Among the possible glycopyranosyl structures are glucose, mannose, galactose, gulose, allose, altrose, idose, or talose. Among the furanosyl structures, the preferred ones are derived from fructose, ribose, arabinose or xylose. Among preferred diglycosides are sucrose, cellobiose, maltose, lactose, trehalose, gentiobiose, and melibiose. Among the triglycosides, the preferred ones may be raffinose or gentianose.

Where there are linked glycosidic units, i.e., there is a di or polyglycosidic residue, the individual glycosidic rings may be bonded by 1-1, 1-2, 1-3, 1-4, 1-5 or 1-6 bonds, most preferably 1-2, 1-4 and 1-6. The linkages between individual glycosidic rings may be α or β.

Aminosaccharides include glucosamine (e.g. the amine is either in the 1- or 2-position), mannosamine, and galactosamine.

Glycuronic acids include hyaluronic acid, glucuronic and galacturonic acids.

In Formula (II), mono-substituted amino group include monoethylamino or monomethylamino, and examples of the di-substituted amino group include diethylamino or dimethylamino. The expression “cyclic-substituted amino group” refers to a 4- to 7-membered ring and examples thereof include azetidinyl, pyrrolidinyl, piperidinyl, morpholinyl, piperazinyl and homopiperazinyl (hexahydro-1H-1,4-diazepin-1-yl). More particularly, the substituent Z means, for example, 4-methyl-1-piperazinyl, 1-piperazinyl, 1-pyrrolidinyl, 3-hydroxy-1-pyrrolidinyl, 1-piperidinyl, 4-hydroxy-1-piperidinyl, 3-hydroxy-1-piperidinyl, 4-morpholinyl, 4-(2-hydroxyethyl)piperazinyl, 3,5-dimethyl-1-piperazinyl, 4-dimethylamino-1-piperidinyl, homopiperazinyl, 1-pyrazolidinyl, 2-methyl-1-pyrazolidinyl, N-(2-hydroxyethyl)amino, N-(2-hydroxyethyl)-N-methylamino, hydrazyl, and methylhydrazyl. See U.S. Pat. No. 4,382,892. A preferred compound of Formula (II) is ofloxacin.

Other quinolones and penicillins that may be prepared in the form of carboxylic acid glycuronides, glycosamides and glycoside esters include, but are not limited to ciprofloxacin, enoxacin, gatifloxacin, levofloxacin, lomefloxacin, moxifloxacin, norfloxacin, ofloxacin, pefloxacin, sparfloxacin, nalidixic acid, trovafloxacin, penicillin, cephalexin, carbenicillin indanyl and cephradine. Where the drug is an amide, the corresponding carboxylic acid may be used.

The compound of this invention can form an acid addition salt with an inorganic or organic acid such as hydrochloric acid, sulfuric acid, methanesulfonic acid and the like.

Especially preferred compounds include the glucose and glucuronic esters of ofloxacin as well as the glucosamide of ofloxacin and the pharmaceutically acceptable salts thereof.

Esters of the compounds of the invention include esters of any free hydroxy groups on the glycose, glycosamine and glycuronide. Such esters include the group R₄—C═O)—, wherein R₄ is as defined above. Preferably such esters are acetate groups which also serve as a protecting group in the condensation reaction.

The carboxylic acid glucuronides may be obtained by condensation of a blocked sugar epoxide with a quinolone (e.g. ofloxacin), penicillin or analog thereof according to the methods disclosed in U.S. Pat. No. 5,633,357. The glycosamides may be prepared by condensation of a protected glycosamine with a quinolone (e.g. ofloxacin), penicillin or analog thereof in the presence of a condensation agent such as dicyclohexyl carbodiimide, DMT-MM or similar agent. The glycose may be protected in 2,3,4,6-positions, e.g. as an acetate, and the anomeric position may be activated as a halide or chloroimidate and then reacted with the carboxylic acid to form the glycoside. The protecting groups may be removed, e.g. by selective deacetylation by using the basic hydroxy resins.

The compounds of the present invention are particularly useful for treatment of infections caused by most facultative gram-negative rods, and have fair activity against staphylococci, and variable to poor activity against streptococci. They are particularly active against P. aeruginosa. Particular diseases and conditions that may be treated with the compounds of the invention include urinary tract infections, infectious diarrhea, systemic gram negative infections, bacterial gastroenteritis, enteric fever, osteomyelitis, gonoccocal infections, amebiosis, bronchitis, complicated skin and soft tissue infections, pneumonia sinusitis, acute maxillary, Streptococcus pneumoniae infection, urinary tract infection and chronic otitis extema in adults.

Particularly preferred routes of administration of the compounds of the present invention are per os, such as elixirs, tablets and capsules, as exemplified below, and by i.v. administration.

More generally, the compounds of the present invention can be administered in any appropriate pharmaceutically acceptable carrier for oral administration since the compounds are biologically active upon oral administration. The compounds of the invention may also be administered in any appropriate pharmaceutical carrier for parenteral, intramuscular, transdermal, intranasal, buccal or inhalation administration. They can be administered by any means that treat or ameliorate the conditions and diseases described herein.

The dosage administered will depend on the age, health and weight of the recipient, kind of concurrent treatment, if any, frequency of treatment and the nature of the effect desired. An exemplary systemic daily dosage is about 0.1 mg to about 500 mg. Normally, from about 1.0 mg to 100 mg daily of the compounds, in one or more dosages per day, is effective to obtain the desired results. One of ordinary skill in the art can determine the optimal dosages and concentrations of active compounds with only routine experimentation.

The compounds can be employed in dosage forms such as tablets and capsules for oral administration. Such dosage forms may comprise well known pharmaceutically acceptable carriers and excipients. In a preferred embodiment, the dosage forms comprise cyclodextran and/or other saccharides and/or sugar alcohols. The compounds may also be formulated in a sterile liquid for formulations such as solutions (e.g. in saline) or suspensions for parenteral use. A lipid vehicle can be used in parenteral administration. The compounds could also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Remington's Pharmaceutical Sciences, 18^{th} Edition, Gennaro et al. (eds.), 1990, exemplifies methods of preparing pharmaceutical compositions.

The compounds may also be employed in fast dissolving dosage forms, as described in U.S. Pat. No. 6,316,027, comprising the compounds of the invention, water, gelatin and other ingredients.

Topical formulations for transdermal, intranasal or inhalation administration may be prepared according to methods well known in the art. For topical administration, the compounds may be applied in any of the conventional pharmaceutical forms. For example, the compounds may be administered as part of a cream, lotion, aerosol, ointment, powder, drops or transdermal patch. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 5% by weight, preferably, 0.001 to 0.5% by weight, more preferably, 0.01 to 0.25% by weight of the active compounds.

The compounds of the invention are substantially pure. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, although the invention is not intended to be so limited.

Having now generally described this invention, the same will be understood by reference to the following examples which are provided herein for purposes of illustration only and are not intended to be limiting unless otherwise specified.

### EXAMPLE 1

### Synthesis of N-(p-methoxybenzylidene)-D-glucosamine

D-glucosamine hydrochloride (215 g; 1 mole) was dissolved in sodium hydroxide solution (1N; 1 liter) and p-anisaldehyde (122 mL) was added. The solid product obtained was filtered off and dried. The product (250 g) had a melting point of 165° C. in accordance with the literature.

### Synthesis of N-(p-methoxy-benzylidene)-1,3,4,6-tetra-O-acetyl-D-glucosamine

The p-anisylidene derivative obtained above (250 g) was dissolved in pyridine (1.25 mL) and acetic anhydride (750 mL) was added slowly at room temperature. The mixture was stirred for 12 hours at room temperature and the clear solution was poured into crushed ice/water mixture (5 liters) and filtered. The precipitate was filtered off and crystallized from methanol (270 g). The product had a melting point of 180-1° C. in accordance with the literature.

### Synthesis of 1,3,4,6-tetra-O-acetyl-D-glucosamine Hydrochloride

To a boiling solution of tetra-O-acetyl-p-anisylidene derivative (150 g) obtained as above in acetone (750 mL) was added hydrochloric acid (SN; 62.5 mL). After stirring the mixture mechanically for 15 minutes, the product was isolated by cooling and adding ether (100 mL) to facilitate complete precipitation. The precipitate was filtered and washed once with ether and dried (100 g; m.p.=230° C.).

### Synthesis of 6-(2′)-glucosylamino (+)-9-fluoro-2,3-dihydro-3-methyl-10-(4-methyl-1-piperazinyl)-7-oxo-7H-pyyrido [1,2,3,-de]-1,4-benzoxazine-6-carboxamide (Glucosamido-Ofloxacin)

Glucosamine hydrochloride (950 mg; 4.4 mMol) was dissolved in water (10 mL) and sodium bicarbonate (380 mg; 4.4 mMol) was added. The solution stirred for few minutes and lyophilized. Dried glucosamine was suspended in dimethylformamide (20 mL) and ofloxacin (1.45 g; 4 mMol) was added at room temperature. Dicyclohexyl carbodiimide (900 mg; 4.4 mMol) was added. The resulting solution was stirred for 16 hours at room temperature. The precipitate was filtered off and the dimethylformamide soluble portion was lyophilized and chromatographed using methanol-dichloromethane mixtures over silica gel. The desired product was obtained as a gummy solid (850 mg).

Proton NMR spectrum in D₂O: δ (1.6; broad singlet; 3-H; methyl); δ (2; singlet; 3-H; N-methyl); δ (2.6; broad singlet; 4-H; N—CH₂); δ (3.4; broad multiplet; 4-H; N—CH₂); δ (4.0-4.8; complex multiplet; 9-H; glucosyl-H and C—H of ofloxacin); δ (5.3; doublet; 1-H; glucosyl-H) and δ (8.5; singlet; Aromatic-H; 1-H).

### Synthesis of 6-[1′,3′,4′,6′-tetra-O-acetyl-glucosyl-2′-amino (+)-9-fluoro-2,3-dihydro-3-methyl-10-(4-methyl-1-piperazinyl)-7-oxo-7H-pyyrido[1,2,3,-de]-1,4-benzoxazine-6-carboxamide (tetra-O-acetyl-Glucosamido-Ofloxacin)

1,3,4,6-tetra-O-acetyl-glucosylamine hydrochloride (1.8 g; 4.4 mMol) suspended in dimethylformamide was added sodium bicarbonate (380 mg) and stirred at room temperature for an hour to dissolution. Ofloxacin (1,45 g; 4.4 mMol) was added followed by dicyclohexylcarbodiimide (900 mg; 4.4 mMol). The solution was stirred for 16 hours at room temperature. The precipitate was filtered and discarded. The clear solution was lyophilized and crystallized from ether (1.2 g).

Proton NMR spectrum in CDCl₃: δ (1.65; broad singlet; 3-H; methyl); δ {2-2.6; complex overlap of singlets; 19-H; N-methyl, N—CH₂ (4H) and (12H)acetyl}; δ (3.4; broad multiplet; 4-H; N—CH₂); δ (4.0-5.2; complex multiplets; 10-H; glucosyl-H and C—H of ofloxacin); and δ (8.5; singlet; Aromatic-H; 1-H) in accordance with the structure.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

1. A compound which is a carboxylic acid glycuronide, glycosamide or glycoside ester of a quinolone, penicillin or analog thereof, or salt thereof.

2. The compound of claim 1, wherein the compound has the Formula (II): 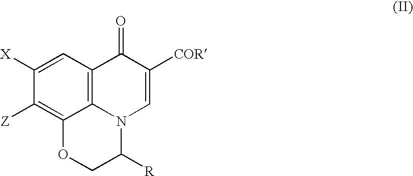
wherein X is a halogen atom, R is a hydrogen atom or an alkyl group having from 1 to 6 carbon atoms, R′ represents the residue of a glycuronic acid, glycosamine or glycoside and Z represents (1) a mono-alkylamino or di-alkylamino group or (2) a cycloamino group selected from the group consisting of azetidinyl, pyrrolidinyl, piperdinyl, morpholinyl, piperidinyl, homopiperazinyl, thiamorpholinyl and pyrazolidinyl, each of which amino groups may be substituted with a hydroxyl group, an alkyl group having 1 to 6 carbon atoms, an amino group, a hydroxyalkyl group having 1 to 6 carbon atoms or a mono- or di-alkylamino group having 1 to 6 carbon atoms in each alkyl group.

3. A compound as in claim 2, wherein Z represents a mono-substituted amino group selected from monoethylamino and monomethylamino, a di-substituted amino group selected from diethylamino and dimethylamino, and a cyclic-substituted amino group comprising a 4- to 7-membered ring selected from azetidinyl, pyrrolidinyl, piperidinyl, morpholinyl, piperazinyl and homopiperazinyl.

4. A compound as in claim 2, wherein R is methyl and Z is 4-methyl-1-piperazinyl.

5. A compound as in claim 2, wherein R is methyl and Z is 4-hydroxy-1-piperazinyl.

6. A compound as in claim 2, wherein R is methyl and Z is 3-hydroxy-1-pyrrolidinyl.

7. A compound as in claim 2, wherein R is methyl and Z is homopiperazinyl.

8. The compound of claim 1 which is glucosamido-ofloxacin.

9. The compound of claim 1, which is a glycose ester containing 1-20 glycose units.

10. The compound of claim 1, wherein said compound is a monoglycose.

11. The compound of claim 6, wherein said glycose unit is a glucose.

12. The compound of claim 1, wherein said compound is a glucuronic acid ester.

13. A pharmaceutical composition comprising the compound of claim 1 and a pharmaceutically acceptable carrier.

14. A method for the treatment or amelioration of a pathogenic infection in an animal, comprising administering to an animal in need thereof an effective amount of the compound of claim 1.

15. The method of claim 14, wherein said compound is administered as part of a pharmaceutical composition comprising a pharmaceutically acceptable carrier therefor.

16. The method of claim 14, wherein said animal is a human.

17. The method of claims 16, wherein said compound is glucosamido-ofloxacin.

18. The method of claim 14, wherein said pathogenic infection is a bacterial infection.

19. The method of claim 14, wherein said pathogenic infection is a fungal infection.
